(19) United States
(12) Patent Application Publication
(10) Pub. No.: US 2022/0196266 A1
(43) Pub. Date: Jun. 23, 2022

(54) HOME AIR STERILIZATION UNIT FOR USE WITH AN HVAC

(71) Applicant: Steven M. Solseth, Oceanside, CA (US)

(72) Inventor: Steven M. Solseth, Oceanside, CA (US)

(21) Appl. No.: 17/692,379

(22) Filed: Mar. 11, 2022

Related U.S. Application Data
(63) Continuation-in-part of application No. 16/932,535, filed on Jul. 17, 2020.
(60) Provisional application No. 63/101,203, filed on Apr. 21, 2020.

(51) Int. Cl. F24F 8/10; F24F 8/22; A61L 9/20

(52) U.S. Cl. CPC F24F 8/10; F24F 8/22; A61L 2209/14; A61L 2209/16; A61L 9/20

(57) ABSTRACT

An indoor air sterilizing system, equipped with at least one UVC radiation source and at least one HEPA filter is described. The system, embodied in a unit, is equipped with a filter frame and a fixture configured to mount and contain a UVC radiation source. The UVC radiation source is connected to a power source via at least one wire. The filter frame, disposed within a housing, is configured to securely hold a HEPA filter in close proximity to the UVC radiation source contained within the fixture. The fixture is held in place within the housing as well. The housing is designed to be affixed to a HVAC return to facilitate sterilization of all of the air within the structure over time in circulation. The housing is affixed to the return via sheet metal screws.

### CONTINUITY

This application is a continuation-in-part of non-provisional patent application Ser. No. 16/932,535 filed on Jul. 17, 2020, and to provisional patent application No. 63/101,203, filed on Apr. 21, 2020 and priority is claimed thereto.

### FIELD OF THE PRESENT INVENTION

The present invention relates to the field of indoor environmental control systems and solutions, and more specifically relates to a new form of air sterilizer configured to sanitize and sterilize the air of a structure via a unique combination of UV radiation and HEPA filter(s) as integrated into an existing HVAC air handler filtration system.

### BACKGROUND OF THE PRESENT INVENTION

In a typical home, the only form of air cleaning is one average quality air filter located in the HVAC air handler. Most of these will not eliminate all the allergens, bacteria, mold, dust, pollens, spores and other unhealthy particulates in the air. More importantly none of these air filters will eliminate the novel Coronavirus. Operating rooms and ICU COVID-19 wards are sterilized using a combination of HEPA filters and UVC lights. Ideally, people should be able to utilize this hospital technology and apply it to homes in the hopes of achieving the same sterilized air by utilizing this type of technology.

If there were a solution present on the market that incorporated UVC radiation in addition to a high-quality HEPA filter for use with a household central HVAC system, viral load inside structures could be dramatically reduced, and the overall quality of air within the structure would be vastly improved. Such a solution preferably positions the UVC radiation source in close proximity to the filter to maximize efficacy. As such, the arrangement of the components within a housing is critical.

Thus, there is a need for a new form of housing and filter apparatus, equipped with a means of emitting UVC radiation in close proximity to the filter, to sufficiently eradicate airborne pathogens while simultaneously substantially reducing airborne particulate counts within a structure. Such an apparatus preferably exhibits a housing having a first frame for containing a fixture to supply UVC radiation and a second frame for containing a high-quality HEPA filter.

### SUMMARY OF THE PRESENT INVENTION

The present invention is an air sterilization unit configured to utilize both UVC radiation and high-quality filtration to cleanse the air of a structure from airborne particulates and pathogens.

As such, the present invention relates to the sterilization of air by combining the benefits of utilizing both a High-Efficiency Particulate Air (hereinafter “HEPA”) filter and an Ultraviolet germicidal bulb (hereinafter “UVC light”), capable of trapping and penetrating unhealthy air particulates and eliminating airborne novel Coronavirus exposed to UVC light. A Base Frame (“A”), referenced as a fixture, is designed to hold a centered UVC light and power cord installed in a residential Heating, Venting and Air Conditioning (hereinafter “HVAC”) Return. A separate Frame (“B”), referenced as a filter frame, designed to accept a HEPA filter, is securely held in place and attached to the Base Frame A with two (2) metal hinges.

The present invention relates to the sterilization of air in a residence by using scientifically proven methods as used in hospital rooms, clean rooms, etc. Properly installed, the unit is configured to eliminate 99% of allergens, bacteria, mold, spores, and pollen, while dramatically reducing the risk of novel Coronavirus exposure.

It is therefore an object of this invention to provide an innovative Home Air sterilization unit.

It is a further object of this invention to provide an Air Sterilization Unit that utilizes the health benefits of a HEPA filter and technology proven to eliminate the novel Coronavirus, allergens, bacteria, mold, dust, pollens, spores and other unhealthy particulates in the air by exposure to UVC light.
the Base Frame A, referenced as a fixture, is configured to accept the UVC light and power cord, is attached to the HVAC return with sheet metal screws, and
the HEPA Filter is held within Frame B, referenced as a filter frame, which is held in the proper position by two (2) metal hinges.
Installation of the present invention does not require modification to any existing HVAC components other than the return installation.
The system of the present invention works on a continuous basis for as long as the HVAC unit is engaged.
The present invention permits the maximum amount of air to flow with a minimum amount of blockage occurring during use.
The present invention presents a solution which does not require excessive power.
The present invention preferably has no moving parts and requires very little maintenance to maintain efficiency.
Additionally, the present invention is easily installed in an existing HVAC return, which has air purification available on demand at the thermostat control unit.
The present invention also adds HEPA benefits by inserting the Filter Frame Unit in the return itself.

The following brief and detailed descriptions of the drawings are provided to explain possible embodiments of the present invention but are not provided to limit the scope of the present invention as expressed herein this summary section.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 exhibits a bottom view of the base support frame of the present invention in an assembled state.

FIG. 2 depicts a front view of the air filter frame of the present invention.

FIG. 3 shows the UVC radiation source and fixture of the present invention in relation to its position proximal to the air filter of the present invention as seen from the side.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention is an air sterilization apparatus and system configured to facilitate the sterilization of air within a structure so as to remove any and all airborne pathogens, pollen, dust, and other particulates. The present invention exhibits a housing (10) equipped with a filter frame (20) disposed within the housing. The filter frame (20) is configured to contain a certified HEPA filter specifically sized to the dimensions of the filter frame (20). A fixture (30), present adjacent and proximal to the filter frame (20) is disposed within the housing (10). The fixture (30) is configured to connect a UVC radiation source (40) to an electric current via a power source (50) via at least one wire (55).

By combining Frame A, designed to properly position the fixture (30) for the UVC radiation source (40), with a second Frame B, referenced as the filter frame (20), which holds a HEPA filter securely in place, this system of the present invention duplicates the sterilizing technology conventionally found in hospital settings. Locating the sterilizing unit of the present invention in the HVAC return ensures that all household air will pass through and be treated. While it cannot be guaranteed that the structure will contain 100% totally virus-free, sterilized air, this system provides the most advanced technology currently available.

The location of a HEPA filter that traps allergens, bacteria, dust, spores and pollen in the return will facilitate keeping the supply line between the return and the air handler uncontaminated.

The Far UVC light, as provided by the UVC radiation source (40) of the present invention, has proven successful in eliminating both the SARs and MERs viruses by destroying their ability to reproduce. The same results are being achieved in fighting the novel Coronavirus in laboratories and hospitals across the country. The Far UVC light cannot cause damage by penetrating the outer layer of the skin or eyes and thus is a major advance over the UVA and UVB lights. Research has shown that far UVC light can eradicate airborne viruses without harming people.

This invention features a method of sterilizing air and eliminating viruses in a home by installing the Frame units, with the UVC light and HEPA filter in place in the HVAC return. This invention is a result of the realization that current technology of utilizing both a HEPA filter and the UVC light to sterilize an operating room could be applied to a home and give similar results. By designing two separate frames and connecting them to form one (1) unit, both the filter and UVC light will properly function together. Installation in the HVAC return ensures all household air will pass through the system continuously.

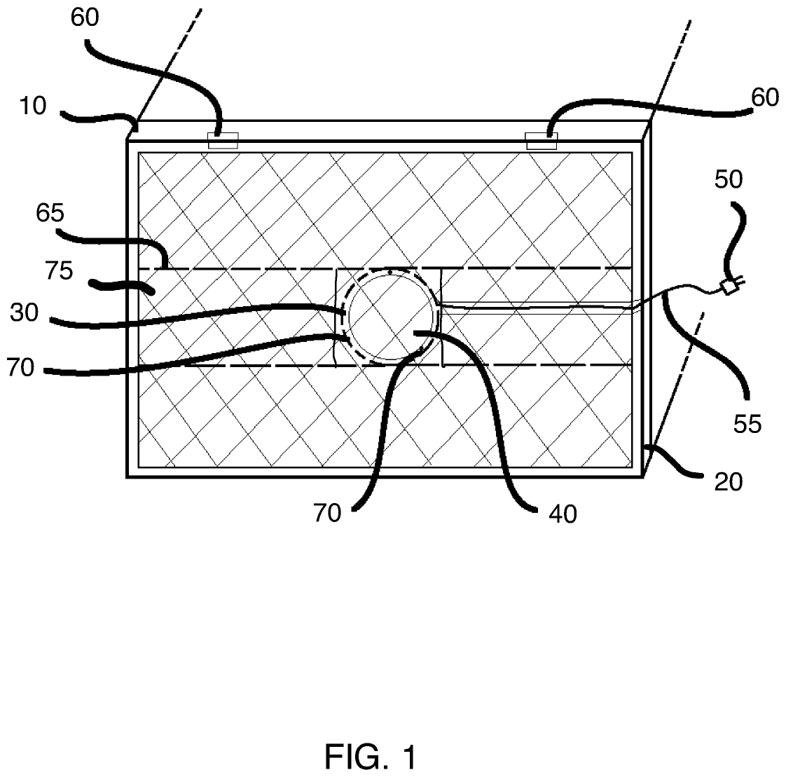

With respect to FIGS. 1-3, FIG. 1 shows a bottom view of the base support frame (A), referenced as the fixture (40) which positions the UVC light centered on a support bar (50). Cut-out areas (75) allow air passage within the housing (10). The housing (10) is attached to the HVAC return with four (4) sheet metal screws.

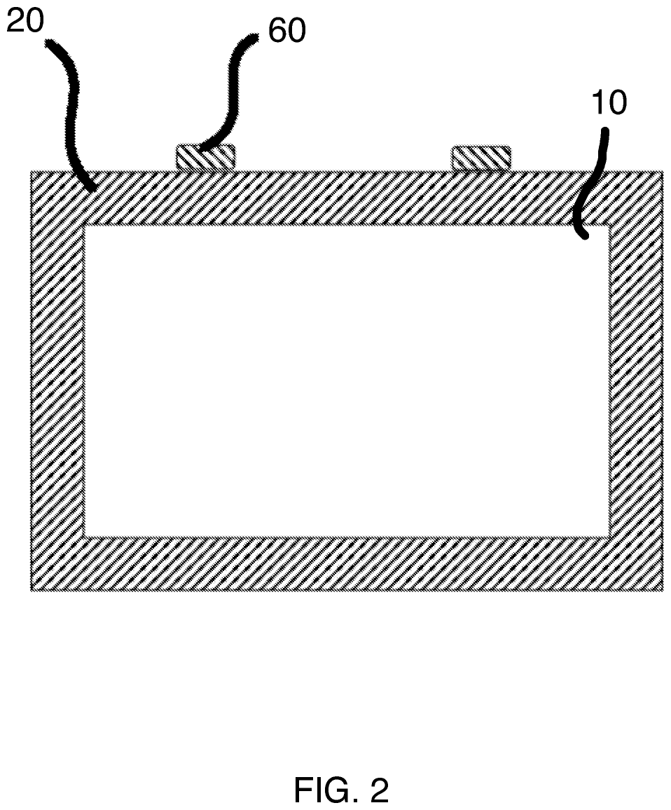

FIG. 2 shows a front view of the filter frame (20) which covers the side and edges of a HEPA filter. Two hinges (60) facilitate the attachment of the filter frame (20) to top of the housing (10), as well as opening of the filter frame (20) about the hinges (60) to facilitate access to the filter.

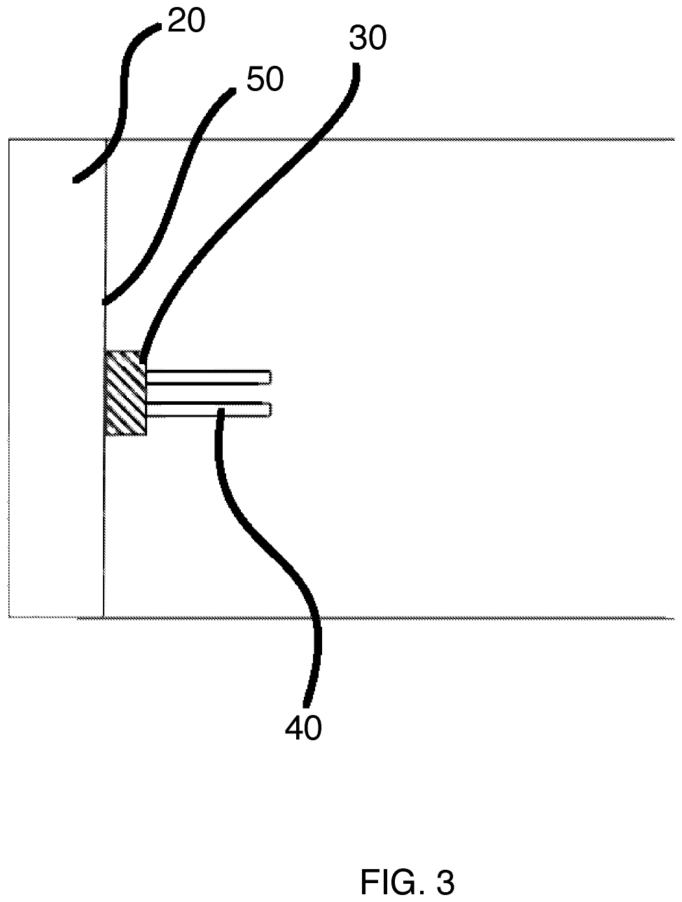

As shown in FIG. 3, the UVC radiation source (40) and fixture (30) are positioned at the center of the Support Bar (65) as shown from the side. The fixture (40) preferably attaches to the support bar (65) using three screws (70). It should be noted that no HEPA filter material is claimed as the filters themselves are not a part of this patent, only the filter frame (20) within the housing (10) configured to contain a conventional HEPA filter.

There are various alternate possibilities regarding installing a UVC radiation source (40) inside an HVAC system, such as within the air handler itself, in the air return, or within additional external air filtration units with a UVC light commercially installed. Additionally, some alternate embodiments of the present invention may require or benefit from the use of a fan, disposed proximal to an intake of the housing (10) to facilitate the flow of air through the unit. The installation of an additional fan or fans can help ensure that air passes through a thick HEPA filter sufficiently.

From the preceding description some embodiments of the present invention become evident: when air particulates are captured in a high quality HEPA filter and then exposed to the UVC light, the air becomes sterilized. The present invention is designed to securely hold the centered position of the UVC radiation source (40) and properly align the Air Filter Frame B to the Base Support Frame A.

Accordingly, it should be understood that embodiments of the present invention are configured to function as designed and are easily assembled. UVC lights and HEPA filters are available from numerous manufacturers and are simple to install.

Furthermore, this air sterilizing system of the present invention utilizes the most advanced technology available today to sterilize air. Applying this technology to a home will provide healthy, virtually virus-free, pure, sterilized air within the structure.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. An air sterilizing system configured to eradicate airborne particulates for use in an HVAC return comprising:
a housing, said housing equipped with a first frame, said first frame configured to contain an HVAC HEPA filter;
a second frame, said second frame containing a fixture, said fixture connected to a power source via at least one wire;
a UVC radiation source, said UVC radiation source disposed within said fixture;
wherein said fixture is disposed behind said first frame within said housing;
wherein said UVC radiation source is configured to cast UVC light towards air which has passed through said HVAC HEPA filter; and
wherein said UVC light penetrates all airborne particulates captured in the HVAC HEPA filter.

2. The air sterilizing system of claim 1, further comprising:
a door, said door connected to said first frame via two hinges.

3. The air sterilizing system of claim 2, wherein said housing is connected to the HVAC return via screws.

4. The air sterilizing system of claim 2, wherein said first frame and said second frame of said housing are made of a plastic composite material, metal, or a combination of these materials.

5. The air sterilizing system of claim 3, wherein said first frame and said second frame of said housing are made of a plastic composite material, metal, or a combination of these materials.

6. A method of sterilizing the air within a structure equipped with a ducted central HVAC system having an HVAC air handler and HVAC return comprising:
affixing a housing to the HVAC return with screws;
wherein the housing is equipped with a first frame containing a HEPA air filter;
wherein the housing is equipped with a second frame having a fixture, the fixture containing a UVC radiation source;
wherein the second frame is disposed behind the first frame;
connecting the UVC radiation source to power via at least one wire;
air within the structure entering the HVAC return through the housing;
the air first passing through the HEPA filter;
the air exiting the HEPA filter;
the air passing by UVC radiation emitted from the UVC radiation source;
the UVC radiation sterilizing the air; and
the air continuing to the HVAC air handler.

7. The method of claim 6, further comprising:
opening a door of the housing about hinges, revealing the HEPA filter;
changing the HEPA filter once it has been soiled by use; and
closing the door of the housing.

8. The method of claim 7, wherein the housing is composed of a plastic composite material, metal, or a combination of these materials.

9. The method of claim 8, wherein the first frame encases all four sides and edges of the HEPA filter, while allowing maximum air passage.

10. The method of claim 9, wherein the UVC radiation and HEPA filter together eliminate all Coronaviruses, allergens, bacteria, mold, pollutants and other unhealthy air particulates from the air.
